(19) United States
(12) Patent Application Publication
(10) Pub. No.: US 2016/0356792 A1
(43) Pub. Date: Dec. 8, 2016

(54) HUMAN BIOMARKER TEST FOR MAJOR DEPRESSIVE DISORDER

(71) Applicant: VINDRAUGA HOLDINGS, LLC, SAN DIEGO, CA (US)

(72) Inventors: John Bilello, Durham, NC (US); Bo Pi, Carlsbad, CA (US); Linda Thurmond, Docham, NC (US)

(73) Assignee: VINDRAUGA HOLDINGS, LLC, SAN DIEGO, CA (US)

(21) Appl. No.: 15/238,570

(22) Filed: Aug. 16, 2016

Related U.S. Application Data
(63) Continuation of application No. 14/211,560, filed on Mar. 14, 2014, now abandoned.
(60) Provisional application No. 61/798,910, filed on Mar. 15, 2013.

(51) Int. Cl. G01N 33/68; G06F 19/20; G06F 19/00; G06F 19/18

(52) U.S. Cl. CPC G01N 33/6893; G06F 19/18; G06F 19/20; G06F 19/345; G01N 2800/304

(57) ABSTRACT

Materials and methods related to diagnosing depression disorders, using a multi-parameter biomarker system and algorithms related thereto.

### CROSS-REFERENCE TO RELATED APPLICATIONS

This application claims benefit of priority from U.S. Provisional Application Ser. No. 61/798,910, filed on Mar. 15, 2013.

### TECHNICAL FIELD

This document relates to materials and methods for diagnosing or assessing Major Depressive Disorder (MDD) in a subject, based on a combination of parameters.

### BACKGROUND

People can live with neuropsychiatric conditions for extended lengths of time. In fact, neuropsychiatric conditions result in more years lived with disability (YLDs) than any other type of condition, accounting for almost 30 percent of total YLDs (Murray and Lopez (1996) Global Health Statistics: A Compendium of Incidence, Prevalence and Mortality Estimates for over 2000 Conditions Cambridge: Harvard School of Public Health). Most care for depression is delivered by general practitioners or primary care physicians (PCPs). However PCP errors in the diagnosis of depression have led to the over and under diagnosis in primary care settings. In a meta-analysis of 50,371 patients across 41 studies PCPs were able to recognize about half (47.3%) of the people who had clinical depression. (Mitchell et al., Lancer, 2009, 374:609-619). At a rate of 21.9%, the positive predictive value was 42.0% and the negative predictive value was 85.8%. This finding suggests that for every 100 unselected cases seen in primary care, there are more false positives (n=15) than either missed (n=10) or identified cases (n=10).

MDD, also known as major depression, unipolar depression, clinical depression, or simply depression, is a mental disorder characterized by a pervasive low mood and loss of interest or pleasure in usual activities. A diagnosis of MDD typically is made if a person has suffered one or more major depressive episodes. MDD affects nearly 19 million Americans annually. The most common age of onset is between 30 and 40 years, with a later peak between 50 and 60 years of age. MDD is a serious disorder not only because of the psychiatric consequences, but also because of the impact on physical health. Depression increases the risk of developing coronary heart disease and, in persons with a prior history of heart attacks, the presence of depression increases the chances of future heart attacks and the chances of dying from a heart attack. Depression also strongly and negatively influences the physical consequences of diabetes and other illnesses. Thus, depression kills not only by increasing the risk of suicide, but also by enhancing the consequences of other common medical illnesses.

### SUMMARY

This document provides reliable, objective methods for diagnosing and/or determining predisposition to MDD. MDD is a heterogeneous illness for which there are currently no effective methods to objectively diagnose, sub-classify, assess severity, or measure the response to treatment. Most clinical disorders, including neuropsychiatric conditions such as MDD, do not arise due to a single biological change, but rather result from an interaction of multiple factors. This document describes the development of a biomarker panel and algorithm for depression that aims to profile diverse peripheral factors that together provide a biological signature of MDD subtypes and aids in diagnosis.

Traditional approaches to biomarkers often have included analyzing single markers or groups of single markers. Other approaches have included using algorithms to derive a single value that reflects disease status, prognosis, and/or response to treatment.

This document is based in part on the identification of methods for using biomarkers and a multi-step algorithm to determine a diagnosis of MDD. The methods can include, for example, selecting a panel of biomarkers related to MDD, obtaining clinical data from subjects for the biomarkers, and applying an optimization algorithm to the clinical data in order to arrive at coefficients for the panel of selected biomarkers. As described herein, the panel was created using the biomarker measurements and coefficients for individuals known to have MDD and those who do not have the condition. In some embodiments of this methodology, for example, algorithms incorporating data from multiple biomarkers in biological samples such as serum or plasma can be developed for patient stratification, identification of pharmacodynamic markers, and monitoring treatment outcome.

In one aspect, this document features a method for assessing the likelihood that an individual has MDD. The method can include (a) identifying groups of biomarkers that may be related to MDD; (b) measuring the level of each of the biomarkers in biological samples from a plurality of subjects, wherein some of the subjects are diagnosed as having MDD and some of the subjects do not have MDD; (c) applying a normalization function to the measured level of each of the biomarkers; (d) applying an optimization algorithm to the measured biomarker levels and calculating coefficients for selected biomarkers within each group; (e) calculating the result of the algorithm for the individual to determine whether the individual is likely to have MDD or is not likely to have MDD. The groups of biomarkers can include two or more inflammatory biomarkers, HPA axis biomarkers, metabolic biomarkers, or neurotrophic biomarkers. The inflammatory biomarkers can be selected from the group consisting of alpha I antitrypsin, alpha 2 macroglobulin, CD40 ligand, interleukin 6, interleukin 13, interleukin 18, interleukin I receptor antagonist, myeloperoxidase, plasminogen activator inhibitor-I, RANTES, and tumor necrosis factor alpha (TNF-α), and soluble TNF-α receptor type II. The HPA axis biomarkers can be selected from the group consisting of cortisol, epidermal growth factor, granulocyte colony stimulating factor, pancreatic polypeptide, adrenocorticotropic hormone, arginine vasopressin, and corticotropin-releasing hormone. The metabolic biomarkers can be selected from the group consisting of adiponectin, acylation stimulating protein, apolipoprotein CIII, fatty acid binding protein, insulin, leptin, prolactin, resistin, testosterone, and thyroid stimulating hormone. The neurotrophic biomarkers can be selected from the group consisting of brain-derived neurotrophic factor, S100B, neurotrophin 3, glial cell line-derived neurotrophic factor, and artemin. The group of biomarkers can consist of alpha-I antitrypsin, apolipoprotein CIII, brain derived neurotrophic factor, cortisol, epidermal growth factor, myeloperoxidase, prolactin, resistin, and soluble tumor necrosis factor receptor type II.

In another aspect, this document features a method for calculating a diagnostic score for MDD based on biomarker measurements and body mass index (BMI). The method can include (a) developing an algorithm for males and an algorithm for females by obtaining measured levels of MDD biomarkers for male and female MDD patients and male and female normal subjects, and applying normalization to each of the measured levels in males and each of the measured levels in females; (b) obtaining a value for a patient's BMI and applying normalization to the BMI; (c) calculating a noontime equivalent value for MDD biomarkers with concentrations that fluctuate in accord with diurnal variation, and applying normalization to each noontime equivalent value; (d) optimizing each algorithm to clinical data and calculating coefficients based on normalized values of the biomarkers that enable segregation of MDD patients from normal subjects: and (e) calculating a MDD diagnostic score for an individual using the algorithm for the individual's gender, wherein the MDD diagnostic score indicates the probability that the individual has MDD.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram depicting steps that can be included in a method for generating a biomarker panel for a particular disease.

FIG. 2 is a diagram of multi-analyte biomarker test development using both a training set and a validation set to optimize the algorithm.

FIG. 3 is a graph plotting an individual MDD patient's normalized biomarker profile in comparison to the population median profile.

FIG. 4 is a pair of graphs plotting MDDSCORE™s for the male and female MDD patients in the validation set.

FIG. 5 is a pair of graphs plotting ROC curves for male and female subjects in the validation set.

FIG. 6 is a graph depicting a simple example of mapping a patient's values in multi-dimensional space.

FIG. 7 is a 3-dimensional hypermap of MDD patients (circles) and normal subjects (squares).

### DETAILED DESCRIPTION

Diagnosis of MDD typically is based on a subject's self-reported experiences and observed behavior. Biobehavioral research, however, is among the most challenging of scientific endeavors, since biological organisms display wide-ranging individual differences in physiology. In particular, the paradigm used for neuropsychiatric diagnosis and patient management is based upon clinical interviews to stratify patients within adopted classifications. This paradigm has the caveat of not including information derived from biological or pathophysiological mechanisms. The biomarker technology and algorithm described herein provide a reliable method to diagnose and/or determine predisposition to depression disorders, and also to assess a subject's disease status and/or response to treatment.

Methods related to multi-analyte diagnostics typically use either a global optimization method in which all the markers (parameters) are used in multivariable optimization to best fit the clinical study results, or use a decision tree methodology. For complex diseases, however, where symptoms overlap and there can be significant variation between stages of disease, a larger number of analytes are required to diagnose or sub-classify patients. In such cases, many parameters need to be taken into account, and the contribution of each parameter (analyte) is small. The present disclosure provides methods for using a biomarker panel and algorithm(s) for reporting the likelihood that an individual has the disease. These methods also include the use of additional clinical information in order to develop a better test that can be optimized for different populations. This current iteration permits the application of the diagnostic test to males vs. females, and accounts for differences in biomarker expression as it is related to gender and body mass index.

In general, the methods described herein are directed to the analysis of multi-analyte diagnostic tests. These methods can be particularly useful with complex diseases, for which it can be difficult to identify one or two markers that will provide enough unique separation between patient sub-groups, e.g., those with a different prognosis or manifestation of disease or, as often occurs with behavioral diseases, distinguishing affected from normal subjects. Multiple markers (e.g., 2, 3, 4, 5, or more than 5 markers) can be used in combination in the presently described methods to provide increased power of a diagnostic test, allowing clinicians to discriminate between patients and prevent confounding co-morbidities from other diseases from interfering with sensitivity and specificity.

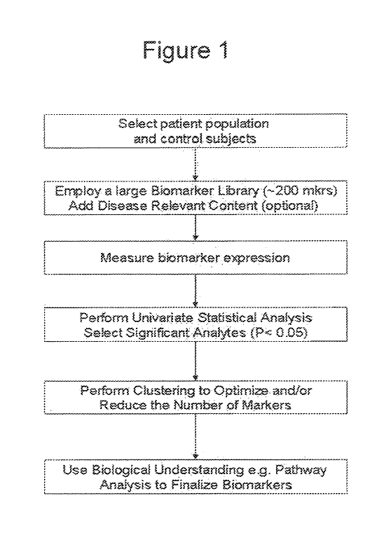

Different groups of markers can be selected based on physiologic/biologic functions related to a disease of interest by use of direct analysis of clinical studies and/or bioinformatics. Using a large library of biomarkers, markers can be grouped according to functional activity that reflects different segments of human physiology and/or biologic processes. Within each group, multiple markers can be used to provide an accurate measurement of the physiologic or biologic changes in each process or system. For analysis of complex diseases, multiple groups can be used for measurement of whole body changes within a particular disease or condition (FIG. 1).

A random generated (e.g., global) optimization that uses all measured markers in all related groups within a body of clinical study data can be used to segregate MDD patients from normal subjects. However, the methods provided herein include optimization of the measured analytes (biomarkers) in each of four functional groups (hypothalamic-pituitary-adrenal (HPA) axis, neurotrophic, metabolic, and inflammatory factors) using data from a clinical study or multiple studies. The results of the analysis of biomarker data from patients and normal subjects can be used in an algorithm to construct a combination parameter or disease score, which functions as an aid to diagnosis. This combination factor essentially is a combined analyte that can be used in calculations of sensitivity and specificity for the test.

In another embodiment, the optimized results for each group can be used to construct a combination parameter that represents the group in the construction of a preliminary multi-dimensional space map (hypermap) of the disease in terms of two or more axes representing functional pathways. Data from multiple studies can be used iteratively to further develop the disease hypermap. The data from individual patients then can be mapped to the disease hypermap in order to take advantage of what is known about other patients whose biomarker profiles fall within the same multi-dimensional space.

Ultimately, the selection of biomarkers is based on the physiology and biology of is the disease, as well as current understanding of biomarker responses within the disease state. Many diseases have shared elements that include inflammation, tissue remodeling, metabolic changes, immune response, cell migration, etc. Certain diseases are associated with pain or neurologic dysfunction, or there may be specific markers that are characteristic of a specific disease (e.g., estrogen receptor levels as an indicator of breast cancer). Biomarkers can be grouped differently, essentially via functional clustering, which can provide more information relative to the pathways involved in physiological dysfunction. In inflammation, for example, markers can include those related to the acute phase response, the cytokine response (e.g., Th1- and Th2-related interleukins), chemokines, and chemoattractant molecules. The following paragraphs set forth exemplary groups of biomarkers.

### Inflammatory Biomarkers

A large variety of proteins are involved in inflammation, and all are open to genetic mutations that can impair or otherwise dysregulate normal expression and function. Inflammation also induces high systemic levels of acute-phase proteins (APP), including C-reactive protein, serum amyloid A, serum amyloid P, vasopressin, and glucocorticoids, which can cause a range of systemic effects. In addition, proinflammatory cytokines and chemokines are involved in inflammation. Table 1 provides an exemplary list of inflammatory biomarkers.

TABLE 1
Exemplary inflammatory biomarkers

| Gene Symbol | Gene Name |
|---|---|
| A1AT | Alpha 1 Antitrypsin |
| A2M | Alpha 2 Macroglobulin |
| CD40L | CD40 ligand |
| IL-6 | Interleukin 6 |
| IL-13 | Interleukin 13 |
| IL-18 | Interleukin 18 |
| IL-1ra | Interleukin 1 Receptor Antagonist |
| MPO | Myeloperoxidase |
| PAI-1 | Plasminogen activator inhibitor-1 |
| RANTES | RANTES (CCL5) |
| TNFA | Tumor Necrosis Factor alpha |
| TNFRII | Soluble TNF α Receptor II |

### HPA Axis Biomarkers

The HPA axis (also referred to as the HTPA axis or the limbic-hypothalamic-pituitary-adrenal axis (LHPA axis)), is a complex set of direct influences and feedback interactions among the hypothalamus, the pituitary gland, and the adrenal (or suprarenal) glands. The interactions among these organs constitute the HPA axis, a major part of the neuroendocrine system that controls reactions to stress and regulates many body processes, including digestion, the immune system, mood and emotions, sexuality, and energy storage and expenditure. Examples of HPA biomarkers include ACTH and cortisol, as well as others listed in Table 2.

TABLE 2
Exemplary HPA axis biomarkers

| Gene Symbol | Gene Name |
|---|---|
| None | Cortisol |
| EGF | Epidermal Growth Factor |
| GCSF | Granulocyte Colony Stimulating Factor |
| PPY | Pancreatic Polypeptide |
| ACTH | Adrenocorticotropic hormone |
| AVP | Arginine Vasopressin |
| CRH | Corticotropin-Releasing Hormone |

### Metabolic Biomarkers

Metabolic biomarkers provide insight into metabolic processes in wellness and disease states. Human diseases manifest in complex downstream effects, affecting multiple biochemical pathways. Proteins and hormones controlling these processes, as well as metabolites, can be used for diagnosis and patient monitoring. Table 3 provides an exemplary of a list of metabolic biomarkers that can be used in the methods described herein.

TABLE 3
Exemplary metabolic biomarkers

| Gene Symbol | Gene Name |
|---|---|
| ACRP30 | Adiponectin |
| APOC3 | Apolipoprotein CIII |
| ASP | Acylation Stimulating Protein |
| FABP | Fatty Acid Binding Protein |
| INS | Insulin |
| LEP | Leptin |
| PRL | Prolactin |
| RETN | Resistin |
| None | Testosterone |
| TSH | Thyroid Stimulating Hormone |

### Neurotrophic Factors

Neurotrophic factors are a family of proteins that are responsible for the growth and survival of developing neurons and the maintenance of mature neurons. Neurotrophic factors have been shown to promote the initial growth and development of neurons in the central nervous system (CNS) and peripheral nervous system (PNS), and to stimulate regrowth of damaged neurons in test tubes and animal models. Neurotrophic factors often are released by the target tissue in order to guide the growth of developing axons. Most neurotrophic factors belong to one of three families: (1) neurotrophins, (2) glial cell-line derived neurotrophic factor family ligands (GFLs), and (3) neuropoietic cytokines. Each family has its own distinct signaling pathway, although the cellular responses that are elicited often overlap. An exemplary list of neurotrophic biomarkers is presented in Table 4.

TABLE 4
Exemplary neurotrophic biomarkers

| Gene Symbol | Gene Name |
|---|---|
| BDNF | Brain-derived neurotrophic factor |
| S100B | S100B |
| NTF3 | Neurotrophin 3 |
| GDNF | Glial cell line derived neurotrophic factor |
| ARTN | Artemin |

From the biomarker groups listed in tables 1-4 above, one can construct a depression diagnostic panel by combining selected markers representative of each group and using an algorithm that provides a decision making parameter. In the case disease diagnosis, a score can be derived that represents the likelihood that a subject has the disease.

### Body Mars Index as a Biomarker

Appetite and weight changes are commonly occurring symptoms of depressive illness. The occurrence of these symptoms may not only be related to depressive mood, but may also be related to body weight. Unipolar depression (MDD) is not a single entity. Approximately one third of MDD cases have been designated as a melancholic sub type, about one third have been designated as atypical with features including hypersomnia and hyperphagia leading to elevated BMI, and the remainder are uncharacterized or represent an indeterminate sub-type. As described herein, BMI can be useful in segregating female MDD patients from normal non-depressed females with a low p value (p≦0.01). Thus, including normalized BMI as a biomarker in the algorithm can increase the ability to segregate a significant segment of the female patient population from normal subjects.

### Clinical Utility of the MDDSCORE™

The utility of the MDDSCORE™ is primarily based on the physiological measurements of biomarkers in the peripheral circulation. This physiologically-based approach differs greatly from diagnosis methods that are subjective in nature. The objective nature of the MDDSCORE™ can provide both the patient and the practitioner with increased confidence in a diagnosis that may initially be based on the practitioner's clinical experience and assessment tools that rely on the patient's ability to accurately report symptoms of their disease.

The tests provided herein are relatively non-invasive, and require only about 10 milliliters of blood. Laboratory measurements of biomarker concentrations can be conducted using readily available, standard assay platforms.

In addition to the final MDDSCORE™, the relative contribution of each biological system to the final score can be assessed. Normalization of biomarker data, discussed below, can provide a direct method for comparison of both individual and multiple biomarkers between patients or within an individual undergoing therapy.

The invention will be further described in the following example, which does not limit the scope of the invention described in the claims.

### Example

### Step 1

### Assembling a Biomarker Panel

To populate a panel (or grouping) of biomarkers for a particular clinical condition, a group of marker candidates is selected that best reflects the disease. In the case of MDD, a group of biomarker candidates was selected from four biological system groups based on their role in MDD. Examples of biomarkers in these four groups are listed in Tables 1 to 4 herein.

Any combination of the markers in each group could have been used to construct a useful biomarker panel for MDD test. The biomarkers that were used were evaluated for their suitability for quantitative measurement, based on the accuracy and precision of the assay in biological fluids (particularly blood, serum, and plasma). In the current example, nine (9) biomarkers were selected from the four broad biochemical domains of inflammation [alpha-I antitrypsin (A1AT), myeloperoxidase (MPO), and soluble TNF receptor type II (sTNFRII)], HPA axis [epidermal growth factor (EGF) and cortisol], neurotrophic or neuroplasticity [brain-derived neurotrophic factor (BDNF)], and metabolic [apolipoprotein CIII (ApoCIII), prolactin (PRL), and resistin (RETN)].

### Step 2

### Assembling a Set of Clinical Data

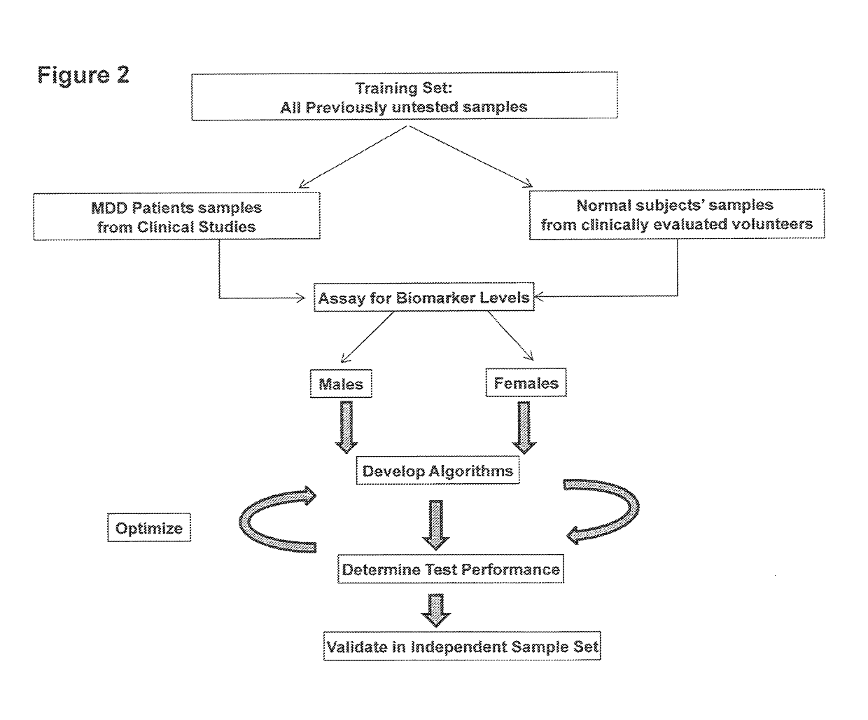

The second step in the processes provided herein typically is to design and collect clinical study data. Biological (clinical) samples were collected from patients having MDD, who typically were diagnosed by known “gold standard” criteria. A set of age- and gender-matched samples also was obtained from normal subjects. Patient samples can be from a group of subjects with different disease states/severities/treatment choices/treatment outcomes. In the present case, patients with different disease severities, duration of disease, treatment options (e.g., different classes of antidepressants), and treatment outcomes (remission, partial remission) were selected. Normal subjects were required to have no history of depression, both personally and in their immediate family members, in addition to being free from confounding diseases. In diagnostic test development, where the goal is prediction, one wants to estimate how accurately a predictive model will perform in practice. One method of test validation, shown in FIG. 2, involves partitioning a sample of data into complementary subsets, performing the analysis on one subset (called the training set), and validating the analysis on the other subset (called the validation set). The size of the training and validation data sets is dependent upon statistical power calculations.

### Step 3

### Biomarker Measurement

Peripheral blood was obtained from patients and normal subjects, and serum prepared using standard methodology was used for biomarker measurement. The measurement of each biomarker was achieved using enzyme-linked immunosorbant assay (ELISA), but other technologies (e.g., mass spectroscopy (MS), liquid chromatography MS (LCMS), turbidimetric assay, or bioassay) also can be used to measure protein and small molecule biomarkers. It also is noted that if nucleic acid biomarkers are to be included in a specific case, such as the determination of the presence or absence of a single nucleotide polymorphism (SNP), other relevant technologies (e.g., PCR) can be utilized.

### Step 4

### Normalization of Biomarker Data

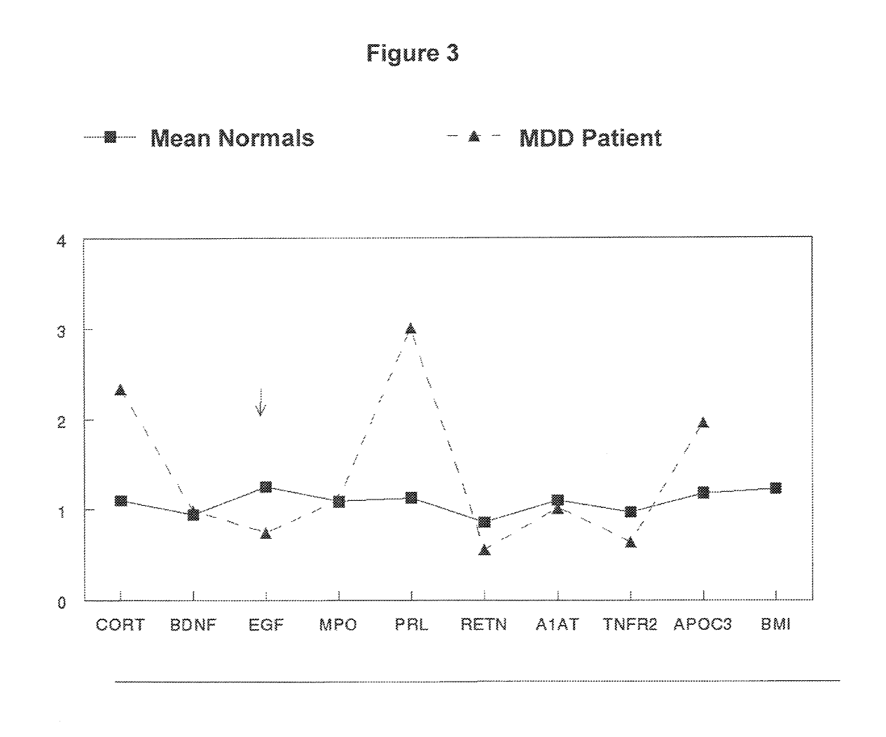

The normalization of biomarker values means adjusting values measured on different scales to a notionally common scale. In the case of the nine biomarkers selected, the relative serum concentration differed widely in magnitude (e.g., from picograms per milliliter to milligrams per deciliter). While such differences may be log transformed, the MDDSCORE™ utilized herein uses a non-traditional approach, in that each biomarker is normalized to the median value of all the samples measured. The advantage of this approach is that it is easy to see the difference between a subject's serum level and that of other normal subjects or MDD patients. By way of example, a subject with a two-fold increase in EGF can easily be identified when compared to other subjects of a similar age, gender and body mass index (FIG. 3). In this example, each raw biomarker concentration, including BMI, was normalized by division with a median derived from a training set of control and MDD samples. Since there are differences in the expression of some biomarkers based on gender, normalization values can be gender-specific. For example, in the training set used for the MDDSCORE™, the median prolactin concentration for females was 9.3 ng/ml and 7.1 ng/ml for males. Normalization also can take diurnal variation into account by using data comparing the serum concentration at various times during the day and developing a “diurnal variation factor” based on the difference between the concentration at the time of draw and the average value at noon. In the MDDSCORE™ example, normalized cortisol was adjusted to noontime equivalent using a table of factors for cortisol diurnal variation based on data collected for multiple MDD patients and normal subjects over a 12 hour span (from 8 AM to 8 PM).

### Step 5

### MDDSCORE™ Algorithm Development and Application

The MDDSCORE™ algorithm was developed to estimate the probability that a patient has MDD. In order to convert the different biomarker values into an effective diagnostic tool for MDD, a bi-logistic formula was used to calculate the probability that a patient has MDD. The normalized values from step 3, Nᵢ, were converted to the odds that a patient has MDD using the formula:

Odds=Exp(b₀+C₁*N₁+C₂*N₂+ . . . +C₁₀*N₁₀)

Where C₁₋₉ are set iteratively, C₁₀ is body mass index and b₀ is a constant derived from the C₁₋₁₀ values with a numeric adjustment, having the value b₀=−(C₁+C₂+ . . . +C₁₀+0.5).

The key optimization feature of the algorithm is reiterative determination of the weighting coefficient. The MDD and normal subject serum samples in a clinical trial data set from step 2 were analyzed and optimized using Receiver Operating Characteristic (ROC) plots to estimate weighting coefficients for each biomarker (Zweig and Campbell, Clin. Chem., 39(8):561-577, 1993). For use in a CLIA laboratory setting, the algorithm is locked, such that the fixed pre-determined coefficient (C₁₋₁₀) is multiplied by the fixed normalized value for each analyte (N₁₋₁₀). Similarly the cortisol diurnal correction, normalization factors, and coefficients for each gender remain constant. In addition to optimizing discrimination between normal and MDD subjects, the coefficients are adjusted to keep a balance between the contributions of the ten markers (nine analytes plus BMI) so the algorithm is responsive to patients whose disease involves a few or all of the pathways.

Once the coefficients were optimized, they were locked and used for subsequent validation studies and ultimately to report patient results.

### Step 6

### Generation of an MDDSCORE™

The MDDSCORE™ roughly corresponds to the increasing likelihood of a patient having a correct diagnosis of MDD. Again, for reporting, the Odds were calculated using the formula:

Odds=Exp(b₀+C₁*N₁+C₂*N₂+ . . . +C₁₀*N₁₀)

Odds were converted to the percent probability that a subject has MDD using the formula:

Percent Probability=(Odds/(1+Odds))

The MDDSCORE™ was calculated using the formula:

MDDSCORE™=100*Odds/(1+Odds)

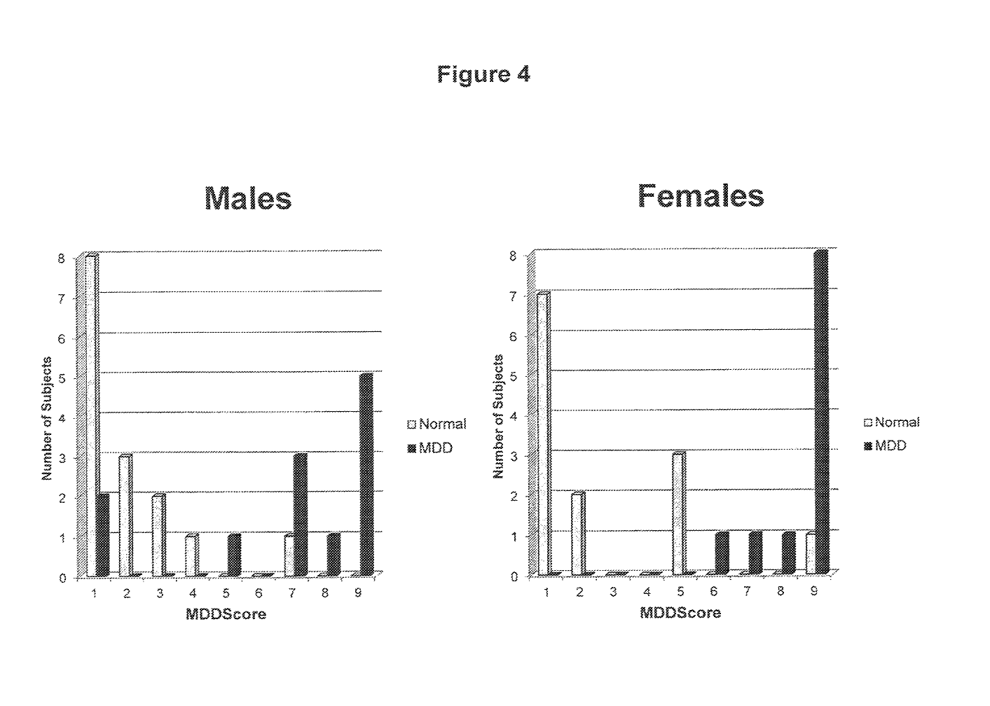

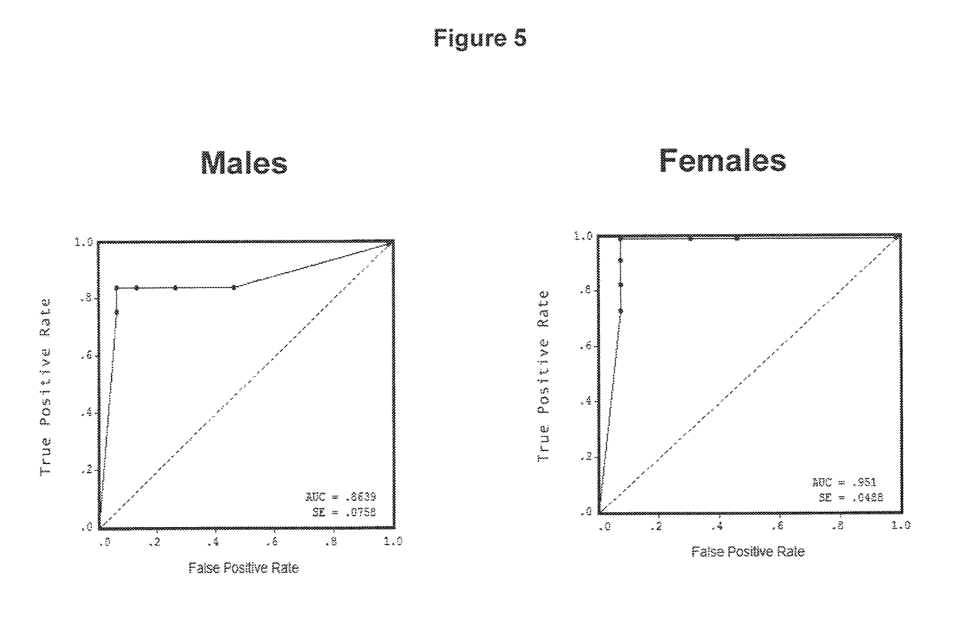

The MDDSCORE™ were binned into nine groups, each group having a score. A score of 1 represented a risk of up to 10% that the patient exhibited a pattern of biomarkers associated with MDD at the time of the blood sampling. Similarly, a score of 5 represented a risk of up to 50%, 8 represented a risk of up to 80%, and 9 represented a risk of up to 90% that the patient's pattern of biomarkers was associated with MDD at the time of sampling. The interpretation of a patient's MDDSCORE™ can be compared to well-characterized clinical patient samples in the form of a histogram. FIG. 4 shows histograms that were developed for male and female subjects upon analysis of the training set and validation sets used in this example. The distribution of MDD patients and normal subjects determined by clinical assessment was primarily bi-modal, with normal subjects having a low MDDSCORE™ (accumulating on the left side of the histogram) and MDD patients having higher scores and accumulating on the right side of the histogram. Since the MDDSCORE™ is based on a combination of analytes, the score can be used to calculate the sensitivity and specificity of the test. The p value for the segregation of MDD male and female patients from normal subjects of the same gender was less than 0.00001. The ROC curves for male and female subjects in the validation set are shown in FIG. 5. The sensitivity and specificity for the male algorithm were 93.3% and 83.3% respectively. Accuracy is measured by the area under the ROC curve. An area of I represents a perfect test; an area of 0.5 represents a worthless test. The AUC for the male gender specific test was 0.864. For the female gender specific test, the sensitivity was 100% and the specificity was 92.3%. The AUC for the female algorithm was 0.951.

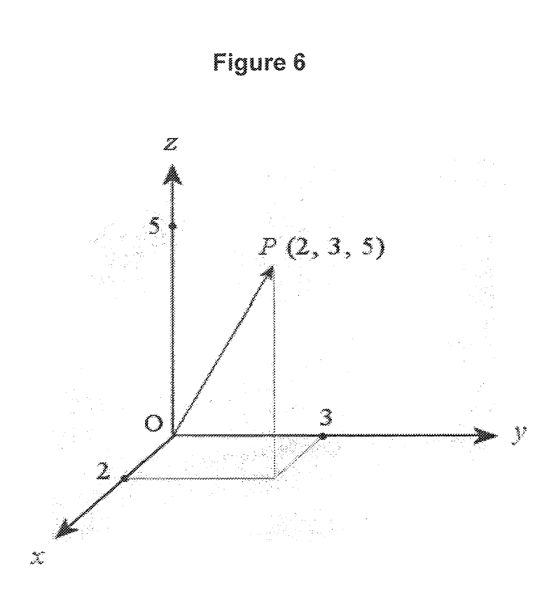

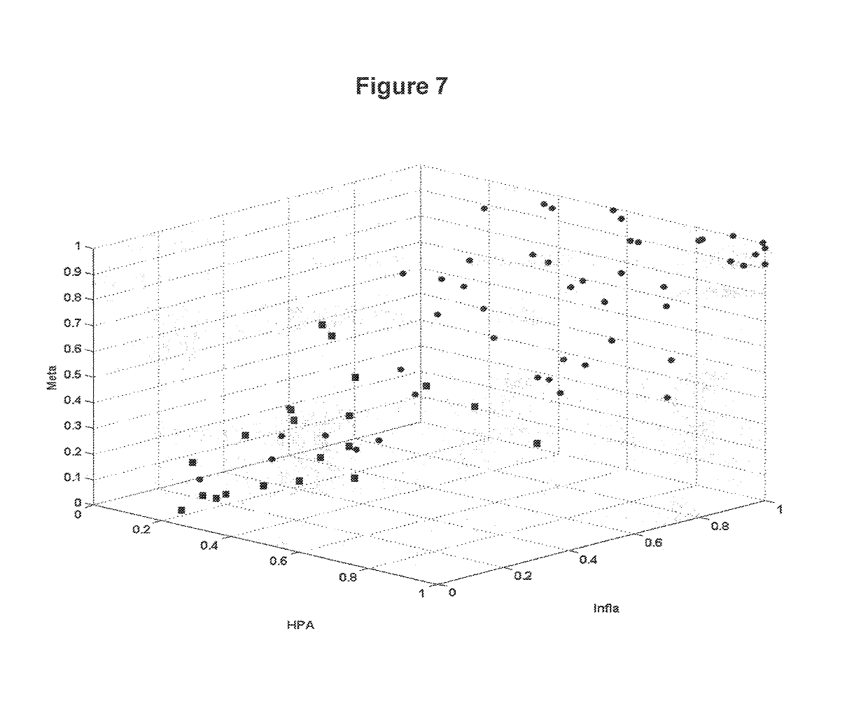

Another embodiment of the methods provided herein is to use the measured biomarker data from the clinical study samples to construct a hyperspace vector for each group of markers. There are several choices of algorithms for constructing hyperspace vectors. A binary logistic regression optimization is used to fit the clinical data with selected markers in each group against the clinical results from “gold standard” diagnosis. Distinct coefficients are used to create hyperspace vectors for each of the pathways. FIG. 6 shows a simple example of a hypermap that was generated by mapping a patient's values in multi-dimensional space. In this example (x, y, and z axes with the values 2, 3, and 5, respectively), the patient's location in multi-dimensional space (P) is described by the values 2, 3, 5. FIG. 7 illustrates the results of applying hyperspace mapping to a set of clinical samples from MDD patients and age-matched control subjects. This hypermap was constructed using data collected from the subjects by measurement and analysis of inflammatory, metabolic, and HPA marker groups. Circles represent patients with MDD, while squares represent normal subjects.

While this document contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

Only a few embodiments are disclosed. Variations and enhancements of the described embodiments and other embodiments can be made based on what is described and illustrated in this document.

What is claimed is:

1. A method for assessing the likelihood that an individual has major depressive disorder (MDD), comprising
(a) identifying groups of biomarkers that may be related to MDD;
(b) measuring the level of each of the biomarkers in biological samples from a plurality of subjects, wherein some of the subjects are diagnosed as having MDD and some of the subjects do not have MDD;
(c) applying a normalization function to the measured level of each of the biomarkers;
(d) applying an optimization algorithm to the measured biomarker levels and calculating coefficients for selected biomarkers within each group;
(e) calculating the result of the algorithm for the individual to determine whether the individual is likely to have MDD or is not likely to have MDD.

2. The method of claim 1, wherein the groups of biomarkers comprise two or more inflammatory biomarkers, HPA axis biomarkers, metabolic biomarkers, or neurotrophic biomarkers.

3. The method of claim 2, wherein the inflammatory biomarkers are selected from the group consisting of alpha 1 antitrypsin, alpha 2 macroglobulin, CD40 ligand, interleukin 6, interleukin 13, interleukin 18, interleukin I receptor antagonist, myeloperoxidase, plasminogen activator inhibitor-I, RANTES, and tumor necrosis factor alpha (TNF-α), and soluble TNF-α receptor type II.

4. The method of claim 2, wherein the HPA axis biomarkers are selected from the group consisting of cortisol, epidermal growth factor, granulocyte colony stimulating factor, pancreatic polypeptide, adrenocorticotropic hormone, arginine vasopressin, and corticotropin-releasing hormone.

5. The method of claim 2, wherein the metabolic biomarkers are selected from the group consisting of adiponectin, acylation stimulating protein, apolipoprotein CIII, fatty acid binding protein, insulin, leptin, prolactin, resistin, testosterone, and thyroid stimulating hormone.

6. The method of claim 2, wherein the neurotrophic biomarkers are selected from the group consisting of brain-derived neurotrophic factor, S100B, neurotrophin 3, glial cell line-derived neurotrophic factor, and artemin.

7. The method of claim 1, wherein the group of biomarkers consists of alpha-1 antitrypsin, apolipoprotein CIII, brain derived neurotrophic factor, cortisol, epidermal growth factor, myeloperoxidase, prolactin, resistin, and soluble tumor necrosis factor receptor type II.

8. A method for calculating a diagnostic score for MDD based on biomarker measurements and body mass index (BMI), comprising:
(a) developing an algorithm for males and an algorithm for females by obtaining measured levels of MDD biomarkers for male and female MDD patients and male and female normal subjects, and applying normalization to each of the measured levels in males and each of the measured levels in females;
(b) obtaining a value for a patient's BMI and applying normalization to the BMI:
(c) calculating a noontime equivalent value for MDD biomarkers with concentrations that fluctuate in accord with diurnal variation, and applying normalization to each noontime equivalent value;
(d) optimizing each algorithm to clinical data and calculating coefficients based on normalized values of the biomarkers that enable segregation of MDD patients from normal subjects; and
(e) calculating a MDD diagnostic score for an individual using the algorithm for the individual's gender, wherein the MDD diagnostic score indicates the probability that the individual has MDD.
